(19) United States
(12) Patent Application Publication
(10) Pub. No.: US 2020/0318096 A1
(43) Pub. Date: Oct. 8, 2020

(54) Nanoparticles to Modulate Transcription-Translation Systems

(71) Applicant: The Government of the United States of America, as represented by the Secretary of the Navy, Arlington, VA (US)

(72) Inventors: Scott Walper, Springfield, VA (US); Igor L. Medintz, Springfield, VA (US); Joyce Breger, Greenbelt, MD (US); Gregory Ellis, Silver Spring, MD (US)

(21) Appl. No.: 16/909,078

(22) Filed: Jun. 23, 2020

Related U.S. Application Data
(63) Continuation-in-part of application No. 15/840,283, filed on Dec. 13, 2017.
(60) Provisional application No. 62/866,047, filed on Jun. 25, 2019.

(51) Int. Cl. C12N 11/18; C12N 11/08; C12N 9/16

(52) U.S. Cl. CPC C12N 11/18; B82Y 5/00; C12N 9/16; C12N 11/08

(57) ABSTRACT

Addition of nanoparticles to a cell-free transcription/translation system significantly enhanced the efficiency of the system.

### CROSS-REFERENCE TO RELATED APPLICATIONS

This application claims the benefit of U.S. Provisional Patent Application No. 62/866,047 filed Jun. 25, 2019, the entirety of which is incorporated herein by reference.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, D.C. 20375, USA; +1.202.767.7230; techtran@nrl.navy.mil, referencing NC 111,209.

### BACKGROUND

Cell-free transcription-translation systems have the potential to make a wide variety of molecular products such as proteins, enzymes, antibodies, small molecules, therapeutics, and the like. Typically, cell-free systems are categorized into either cell extract or recombinant systems. While there are advantages to each type of system, cell extracts are made up of undefined and complex components and recombinant systems are simpler with defined components. One such cell-free system called the “PURE” system (protein synthesis using recombinant elements) has been commercialized by New England Biolabs (NEB) and is called PURExpress. This system contains proprietary components and at least a portion of the enzyme components contain polyhistidine. The major drawbacks of the PURExpress system are the high cost of the kit and that limited options exist for the kit to be optimized or customized by the user.

### BRIEF SUMMARY

In one embodiment, a method of enhancing protein production includes providing a cell-free transcription/translation system; adding nanoparticles to the system; and then performing protein production with the system, wherein the system comprises at least one enzyme having multiple polyhistidine tags and the addition of nanoparticles causes formation of clusters of nanoparticles, such that, on average, each nanoparticle in a cluster is separated from a nearest neighboring nanoparticle by a distance of no more than about one nanoparticle diameter

In another embodiment, a method of producing enzyme activity from a cell-free system comprising: providing a cell-free transcription/translation system comprising at least one enzyme having multiple polyhistidine tags (for example, an enzyme operable as a dimer or tetramer) and nanoparticles; then causing the system to produce an enzyme of interest, and then allowing the enzyme of interest to act on at least one substrate; wherein the system includes at least one enzyme having multiple polyhistidine tags and the addition of nanoparticles causes formation of clusters of nanoparticles, such that, on average, each nanoparticle in a cluster is separated from a nearest neighboring nanoparticle by a distance of no more than about one nanoparticle diameter. In further embodiments, such a method is performed at a site known or suspected to contain a contaminant and the enzyme of interest is effective to degrade the contaminant.

In still another embodiment, a method of site decontamination includes identifying a site known or suspected to contain a contaminant; providing a cell-free transcription/translation system comprising at least one enzyme having multiple polyhistidine tags (for example, an enzyme operable as a dimer or tetramer) and nanoparticles; then causing the system to produce an enzyme of interest effective to degrade the contaminant, and then allowing the enzyme of interest to degrade the contaminant at the site.

### BRIEF DESCRIPTION OF THE DRAWINGS

FIGS. 1 is a schematic illustration showing nanoparticle (NP)-enhanced cell-free reactions. Such in vitro transcription-translation (TX-TL) reactions combine the necessary cellular machinery/components for these biological processes with a nucleic acid template (normally DNA) encoding a protein of interest. The typical TX-TL reaction relies on passive diffusion of these components along with their cognate cofactors and substrates to translate the DNA template into RNA (transcription) and RNA into protein (translation). While effective, assembly of these components to NP surfaces and subsequent macroscale clustering leads to improved performance evidenced by increased reaction durations and increased levels of protein production (as seen in the insert graphs).

FIG. 2 is a transmission electron micrographic (TEM) image showing assembly and clustering of transcription-translation (TX-TL) reactions. Epitope tags on many of the TX-TL reaction components allows for these proteins to be non-covalently attached/coordinated to the surface of NPs. Because a number of these component proteins are multimeric, they present two or more terminal epitope tags thus allowing nanoparticle clustering to occur. This phenomenon reduces diffusional distances and potentially allows for substrate channeling between individual reaction components.

FIGS. 3A and 3B show the effect of NP concentration on reaction enhancement.

FIG. 4 provides data on the enhancement degradation of paraoxon by phosphotriesterase (PTE) produced of on or off nanoparticles.

### DETAILED DESCRIPTION

Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

The terms “semiconductor nanocrystal,” “quantum dot,” and “QD” are used interchangeably herein and refer to an inorganic crystallite of about 1 nm or more and about 1000 nm or less in diameter or any integer or fraction of an integer therebetween, preferably at least about 2 nm and about 50 nm or less in diameter or any integer or fraction of an integer therebetween, more preferably at least about 2 nm and about 20 nm or less in diameter (for example about 2, 3, 4, 5, 6, 7, 8, 9, 10, 11, 12, 13, 14, 15, 16, 17, 18, 19, or 20 nm). QDs are characterized by their relatively uniform nanometer size. A QD is capable of emitting electromagnetic radiation upon excitation (the QD is luminescent) and includes a “core” of one or more first semiconductor materials, with the core optionally surrounded by a “shell” of a second semiconductor material.

The term “nanoparticle” or “NP” as used herein includes the above-mentioned QDs in addition to other nano-scale and smaller particles such as metallic nanoparticles (e.g., nanoparticles comprising Ag, Au, Cu, Pd, Pt, and combinations thereof), carbon nanotubes, proteins, polymers, dendrimers, viruses, and drugs. A nanoparticle has a size of less than about 1 micron, optionally less than about 900, 800, 700, 600, 500, 400, 300, 200, 100, 80, 60, 50, 40, 30, 20, 10, 9, 8, 7, 6, 5, 4, 3, 2, or 1 nanometers. A nanoparticle may have various shapes such as a rod, a tube, a sphere, and the like. Nanoparticles may be made from various materials including metals, carbon (such as carbon nanotubes), polymers, and combinations thereof.

Overview

Described herein is the use of NPs such as quantum dots (QDs) for the attachment of polyhistidine-containing components or products from cell-free transcription-translation systems and how attachment enhances or modulates the cell-free transcription-translation systems.

Polyhistidine tagged enzymes and proteins can self-assemble onto Zn-coated NPs in a rapid, ratiometric, and facile manner. With super-folded green fluorescent protein (sfGFP) as an example product and the PURExpress kit (which has His-tagged components) as an example system, NPs were employed with a cell-free transcription-translation system to increase the rate of product formation. This seems to have been a result of increased enzyme stabilization on NPs, substrate channeling, and the ability of NPs to interact with a number of enzyme species forming enzyme clusters. As cell-free transcription-translation systems can be employed for a variety of functions, so too can the value of NP addition to the reaction be equally variable. In biomanufacturing, NP can improve the efficiency of the transcription-translation process leading to higher levels of protein production. There is also significant interest in cell-free systems as biosensors. Here increased efficiency of reporter synthesis could improve the overall sensitivity, limit of detection, or time of response for the system.

Even in the case of a commercial cell-free translation kit having components that are largely a mystery to the end user, certain things are known regarding the necessary contents. For example, several of the proteins required are multimeric, such as aminoacyl-tRNA synthetases. Thus, in the case of a transcription-translation (TX-TL) kit containing histidine-tagged proteins, these multimeric proteins would be expected to facilitate formation of clusters when contacted with nanoparticles having a suitable metallic surface. (such as Zn-coated nanoparticles) despite the user not necessarily knowing the precise contents of the kit.

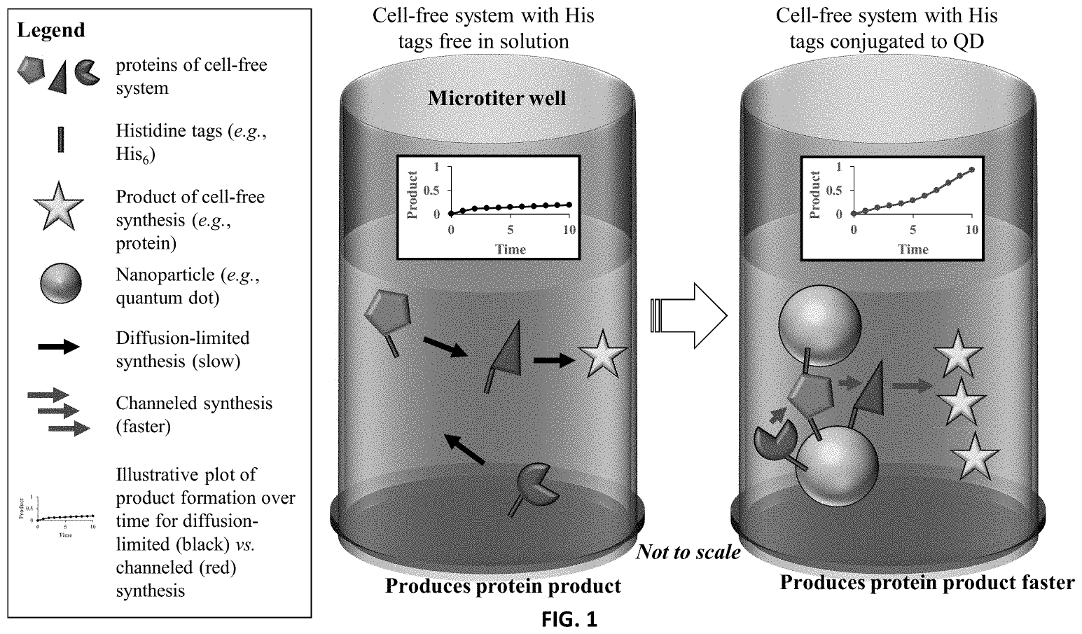

FIG. 1 shows a schematic where in vitro TX-TL reactions combine all of the necessary cellular machinery/components for these biological processes with a nucleic acid template (DNA) encoding a protein of interest. The typical TX-TL reaction relies on passive diffusion of these components along with their cognate cofactors and substrates to translate the DNA template into RNA (transcription) and RNA into protein (translation). While effective, assembly of these components to NP surfaces and subsequent macroscale clustering leads to improved performance evidenced by increased reaction durations and increased levels of protein production (insert graphs for each).

Cell-free production of enzymes enables bio-based sensors, on-demand synthesis of biomolecules, and field-deployable decontamination strategies. NP-clustering of cell-free components lowers diffusional limitations and potentially allows for substrate/product shuttling which in turn leads to more rapid rates of protein synthesis. This translates to improved response times for sensors and catalytic systems.

### EXAMPLES

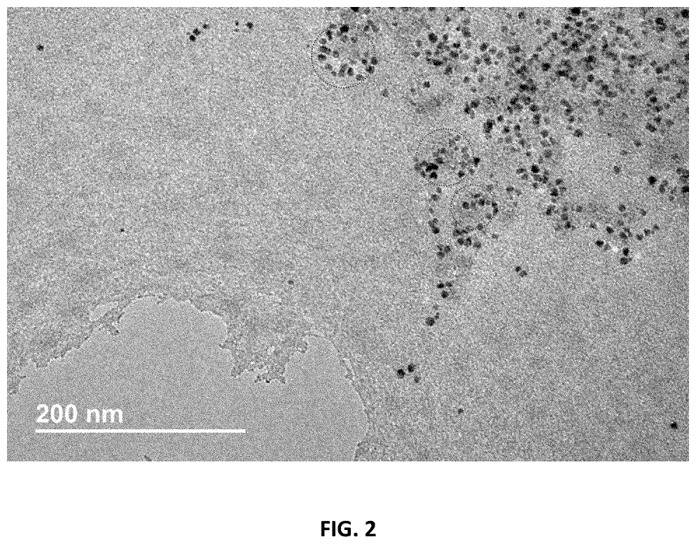

FIGS. 2 is a transmission electron micrographic (TEM) image showing clustering of NP-assembled TX-TL machinery. Epitope tags on many of the TX-TL reaction components allows for these proteins to be non-covalently attached/coordinated to the surface of NPs, in this case quantum dot 625 (QD625). As many TX-TL component proteins are multimeric, many of these proteins will present two or more terminal epitope tags allowing nanoparticle clustering to occur. This phenomenon reduces diffusional distances and potentially allows for substrate channeling between individual reaction components.

The concentrations of components within; proteins, cofactors, substrates, DNA template; are expected to directly affect the overall yield of the reaction. Similarly; the size, morphology, and concentration of NPs added to TX-TL reactions will alter both the reaction kinetics and total protein yield requiring some optimization to determine NP concentration for the desired conditions.

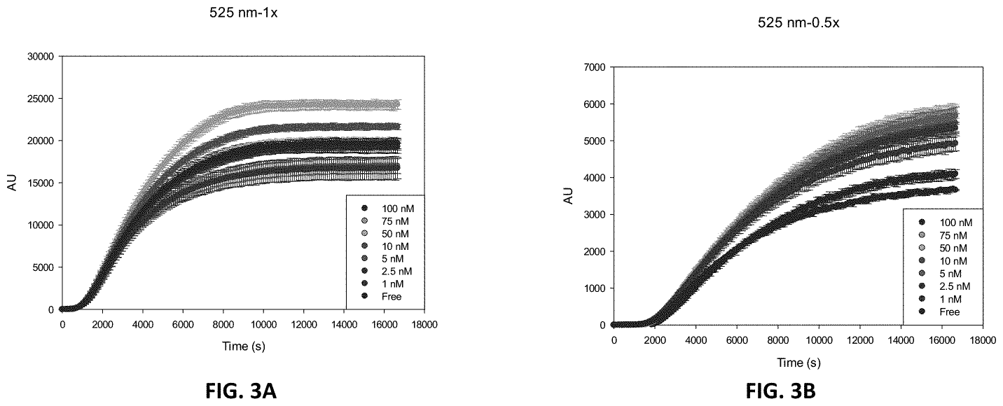

In order to examine concentration dependence, solutions A and B from the PURExpress kit were either undiluted (labeled “1×” as shown in FIG. 3A) or diluted by a factor of two (labeled “0.5×” as shown in FIG. 3B) and incubated in the presence of increasing amounts of either 520 nm QDs. As shown in FIG. 3A, 75 nM of 525 nm quantum dots (QDs) show the greatest enhancement in green fluorescent protein synthesis.

It was surprising and unexpected that the addition of nanoparticles enhanced the transcription/translation activity of this propriety system that necessarily included large protein complexes (RNA polymerase and ribosomes).

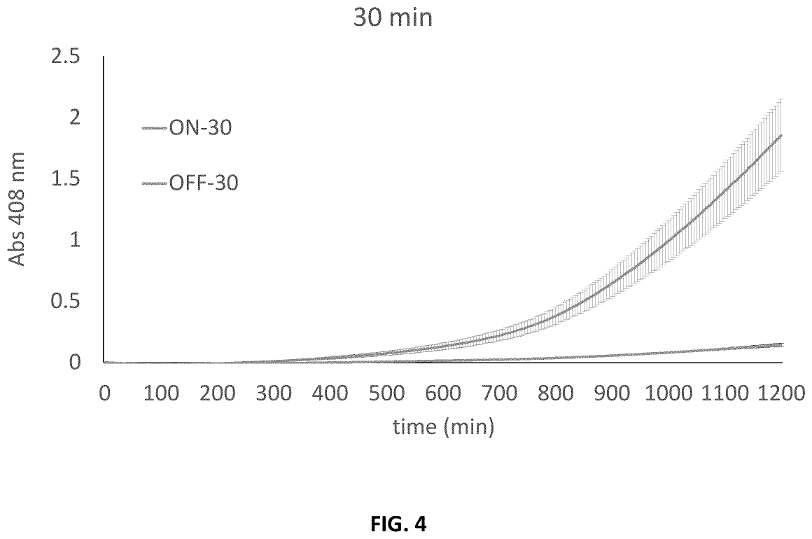

FIG. 4 provides data on the enhancement degradation of paraoxon by phosphotriesterase (PTE) produced on or off nanoparticles. Cell-free transcription-translation reactions were performed in triplicate with the PURExpress in vitro protein synthesis kit (NEB). For nanoparticle coupling, stock solutions of PURExpress (Solution A+Solution B) or PURExpress/QD were allowed to assemble for 30 min on ice. 20 μl of the PURExpress or PURExpress/QD stock solution was added to the wells of a 384 well microtiter plate followed by addition of 100 ng DNA (pY71-PTE). The reaction was incubated at 37° C. and inhibited with 200 μg/ml kanamycin at indicated time points. PTE activity was assayed by addition of 25 μl paraoxon (diethyl 4-nitrophenyl phosphate) diluted (1:1,000) in 2-(cyclohexylamino)ethanesulfonic acid (CHES) buffer (50 mM, pH 8). Enzyme-mediated hydrolysis of the paraoxon substrate to p-nitrophenol was monitored at 408 nm for 20 hr in a plate reader 30° C. (Biotek).

### Further Embodiments

It is expected that this technique could be expanded to a number of other cell-free systems for transcription and/or translation, for example commercial kits from other manufacturers. Preferably, the system used would have at least a portion of the included enzymes and/or factors incorporating functionality (for example, polyhistidine tags) making them amenable to immobilization on nanoparticles or another suitable material.

Other materials, such as DNA nanostructures and gold nanoparticles, have groups that could be used for enzyme immobilization and co-localization of enzymes within an enzymatic cascade.

In additional embodiments, the system includes at least one enzyme having multiple polyhistidine tags (for example, an enzyme operable as a dimer or tetramer), thus enhancing formation of clusters of nanoparticles. For example, nanoparticles in the cluster can be closely associated with one another such that, on average, each nanoparticle is separated from the nearest neighboring nanoparticle by a distance of no more than about one nanoparticle diameter.

Advantages

Utilizing components or products from a cell-free transcription-translation system that contain a polyhistidine-tag to bind to metal-QDs or NPLs to enhance overall performance offers the following advantages:
(1) The technique is operable on uncharacterized or propriety systems including cell-free transcription/translation kits where the concentration and stoichiometry of components is unknown
(2) Metal-QDs or NPLs can be easily functionalized with a wide variety of surface ligands that provide different surface charges, polarities, and steric bulk
(3) Enzymes and other components of cell-free transcription-translation systems can be easily and tightly bound to NP surface through a simple hexahistidine tag, which can be incorporated genetically into the enzymes of interest
(4) The ability to site-specifically locate the hexahistidine tag on the enzyme allows for more uniform orientations of the enzymes on the surface
(5) QDs and NPLs can often enhance the activity of individual bound enzymes
(6) Binding oligomeric enzymes to NPs via hexahistidine tags can stabilize the oligomeric structure at low concentrations and enhance activity
(7) The activity and stability are greatly enhanced when attached to NPs compared to free components at very low concentrations
(8) The colocalization of enzymes on QDs and NPLs allows for substrate channeling, thus further enhancing the kinetics of the reaction
(9) The large surface area and dimensions of QDs and NPLs allows for the conjugation of numerous enzymes to the surface
(10) Enzymes can be stabilized by binding to a QD or NPL surface
(11) Substrates appear to accumulate near QD or NPL surfaces which may further facilitate substrate channeling between multiple bound enzymes
(12) Large, complex enzyme-NP clusters can be formed from the products or components of a cell-free transcription-translation system and may facilitate substrate channeling between enzymes. This is based on the oligomeric enzyme crosslinking the NPs to each other into higher order structures.
(13) Attaching His6-appended cell-free components to NPs/NPLs can stabilize their long-term shelf life in solution and when frozen and/or if lyophilized for later rehydration.

By utilizing NPs to increase the efficiency of a cell-free transcription-translation system, low amounts of the system can be utilized while maintaining product formation, decreasing the overall cost of the system. In embodiments, the addition of nanoparticles allows the kit components to be diluted, resulting in a net cost savings as the kits are relatively expensive compared to the amount of nanoparticles required for effective enhancement.

Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

[1] Lavickova, B., Maerkle, S. J. (2019) A simple, robust, and low-cost method to produce the PURE cell-free sytem, ACS Synth. Biol., 8 (2), 455-462

[2] Li, J., Gu, L., Aach, J., Church, G. M. (2014) Improved cell-free RNA and protein synthesis system, PLOS One, 9 (9), 1-11

[3] Vranish, J. N., Ancona, M. G., Oh, E., Susumu, K., Medintz, I. L. (2017) Enhancing coupled enzymatic activity by conjugating one enzyme to a nanoparticle, Nanoscale 9, 5172-5187

[4] Vranish, J. N., Ancona, M. G., Walper, S. A., Medintz, I. L. (2018) Pursuing the Promise of Enzymatic Enhancement with Nanoparticle Assemblies, Langmuir 34, 2901-2925

[5] Ansari, S. A., and Husain, Q. (2012) Potential applications of enzymes immobilized on/in nano materials: A review, Biotechnology Advances 30, 512-523.

[6] Blanco-Canosa, J. B., Wu, M., Susumu, K., Petryayeva, E., Jennings, T. L., Dawson, P. E., Algar, W. R., and Medintz, I. L. (2014) Recent progress in the bioconjugation of quantum dots, Coordin Chem Rev 263, 101-137.

[7] Breger, J. C., Ancona, M. G., Walper, S. A., Oh, E., Susumu, K., Stewart, M. H., Deschamps, J. R., and Medintz, I. L. (2015) Understanding how nanoparticle attachment enhances phosphotriesterase kinetic efficiency, ACS Nano 9, 8491-8503.

[8 Breger, J. C., Walper, S. A., Oh, E., Susumu, K., Stewart, M. H., Deschamps, J. R., and Medintz, I. L. (2015) Quantum dot display enhances activity of a phosphotriesterase trimer, Chem Commun 51, 6403-6406.

[9] Brown, C. W, Oh, E., Hastman, D. A., Walper, S. A., Susumu, K., Stewart, M. H., Deschamps, J. R., and Medintz, I. L. (2015) Kinetic enhancement of the diffusion-limited enzyme beta-galactosidase when displayed with quantum dots, RSC Adv 5, 93089-93094.

[10] Claussen, J. C., Malanoski, A., Breger, J. C., Oh, E., Walper, S. A., Susumu, K., Goswami, R., Deschamps, J. R., and Medintz, I. L. (2015) Probing the enzymatic activity of alkaline phosphatase within quantum dot bioconjugates, J Phys Chem C 119, 2208-2221.

[11] Es, I., Vieira, J. D. G., and Amaral, A. C. (2015) Principles, techniques, and applications of biocatalyst immobilization for industrial application, Appl Microbiol Biot 99, 2065-2082.

[12] Fu, J. L., Liu, M. H., Liu, Y, Woodbury, N. W, and Yan, H. (2012) Interenzyme substrate diffusion for an enzyme cascade organized on spatially addressable DNA nanostructures, J Am Chem Soc 134, 5516-5519.

[13] Johnson, B. J., Algar, W. R., Malanoski, A. P., Ancona, M. G., and Medintz, I. L. (2014) Understanding enzymatic acceleration at nanoparticle interfaces: Approaches and challenges, Nano Today 9, 102-131.

[14] Sapsford, K. E., Pons, T., Medintz, I. L., Higashiya, S., Brunel, F. M., Dawson, P. E., and Mattoussi, H. (2007) Kinetics of metal-affinity driven self-assembly between proteins or peptides and CdSe—ZnS quantum dots, J Phys Chem C 111, 11528-11538.

What is claimed is:

1. A method of enhancing protein production, comprising:
providing a cell-free transcription/translation system;
adding nanoparticles to the system; and then
performing protein production with the system,
wherein the system comprises at least one enzyme having multiple polyhistidine tags and the addition of nanoparticles causes formation of clusters of nanoparticles, such that, on average, each nanoparticle in a cluster is separated from a nearest neighboring nanoparticle by a distance of no more than about one nanoparticle diameter.

2. The method of claim 1, wherein the cell-free transcription/translation system is a proprietary system where the concentration and stoichiometry of the system including components unknown.

3. The method of claim 2, wherein the system is diluted compared to a recommended system concentration without the nanoparticles.

4. A method of producing enzyme activity from a cell-free system comprising:
providing a cell-free transcription/translation system comprising at least one enzyme having multiple polyhistidine tags (for example, an enzyme operable as a dimer or tetramer) and nanoparticles; then
causing the system to produce an enzyme of interest, and then
allowing the enzyme of interest to act on at least one substrate;
wherein the system includes at least one enzyme having multiple polyhistidine tags and the addition of nanoparticles causes formation of clusters of nanoparticles, such that, on average, each nanoparticle in a cluster is separated from a nearest neighboring nanoparticle by a distance of no more than about one nanoparticle diameter.

5. The method of claim 4, wherein the method is performed at a site known or suspected to contain a contaminant and the enzyme of interest is effective to degrade the contaminant.

6. A method of site decontamination comprising:
identifying a site known or suspected to contain a contaminant;
providing a cell-free transcription/translation system comprising at least one enzyme having multiple polyhistidine tags (for example, an enzyme operable as a dimer or tetramer) and nanoparticles; then
causing the system to produce an enzyme of interest effective to degrade the contaminant, and then
allowing the enzyme of interest to degrade the contaminant at the site.

7. The method of claim 6, wherein the enzyme of interest is phosphotriesterase.
